(19) United States
(12) Patent Application Publication
(10) Pub. No.: US 2024/0395400 A1
(43) Pub. Date: Nov. 28, 2024

(54) Method and Apparatus for Securely Documenting Medical Treatment Regimes

(71) Applicant: Fresenius Medical Care Deutschland GmbH, Bad Homburg (DE)

(72) Inventors: Michael Thorwarth, Bad Homburg (DE); Yizhuo Wang, Bad Homburg (DE)

(21) Appl. No.: 18/687,054

(22) PCT Filed: Aug. 31, 2021
(86) PCT No.: PCT/CN2021/115616

§ 371 Date: Feb. 27, 2024

(51) Int. Cl. G16H 40/67; G16H 20/40; G16H 30/00

(52) U.S. Cl. CPC G16H 40/67; G16H 20/40; G16H 30/00

(57) ABSTRACT

The present disclosure relates to securely documenting medical treatment regimes as part of a performed medical treatment. The techniques described herein involve using at least double authentication of at least two different authorized persons. The present disclosure also relates to apparatuses into which medical treatment regimes can be input, or that automatically detect a medical treatment regime, and with which a confirming endorsement linked to a medical treatment regime can be permanently stored.

### CROSS-REFERENCE TO RELATED APPLICATIONS

The present application is the national stage entry of International Application No. PCT/CN2021/115616, filed on Aug. 31, 2021, the contents of which are hereby incorporated by reference.

### TECHNICAL FIELD

The present disclosure relates to methods and apparatuses for securely documenting medical treatment regimes. Additionally, the present disclosure includes a software-controlled device, a system, a computer program and a computer-readable data medium.

### BACKGROUND

In the clinical field, there is a desire for medical treatments to be documented as accurately and securely as possible. Medical treatments can include any treatments used to treat a chronic or acute illness. Of particular interest are chronic or acute dialysis, which can be performed in a variety of manners. Medical evaluation of the performed treatment by medical staff or research personnel may depend on how accurately performed treatments are documented. Based on these available reliable documented data, medical staff or research personnel can derive further therapy options or research results that can contribute to the safe or improved treatment of the specific illness of an individual patient or an illness in general.

If a single person manually performs this documentation, processing errors can occur, which can jeopardize further therapies or can lead to incorrect conclusions in the research based thereon.

### SUMMARY

The present disclosure relates to documenting medical treatment data securely and accurately.

In an example implementation, a method described herein includes enabling a user interface of a software-controlled device for the input of information relating to medical treatment regimes only if at least one first authentication feature is input into the software-controlled device and the first authentication feature meeting first conditions. The method further includes inputting information relating to a medical treatment regime into the software-controlled device. Additionally, the method includes inputting at least one confirming endorsement and linking the confirming endorsement to the input information. The confirming endorsement is permanently stored only if at least one second authentication feature is input into the software-controlled device, where the second authentication feature meets second conditions. The first conditions include the circumstance of the first authentication feature, which corresponds to an authentication feature from a first group of authentication features authorizing the input of information relating to the medical treatment regime into the software-controlled device, and the first authentication feature being associated with a first person. The second conditions include the circumstance of the second authentication feature corresponding to an authentication feature from a second group of authentication features authorizing the instigation of the permanent storage of the confirming endorsement, and the second authentication feature being associated with a second person.

In some embodiments, operations of the method can be performed by a software-controlled device, a system including at least one medical device designed to treat a patient, a computer program including instructions that, when executed by the software-controlled device, cause the software-controlled device to perform said operations, or a computer-readable data medium upon which the computer program is stored.

A medical treatment regime within the context of the present disclosure can include (but is not limited to): specific methods of treatment such as for example hemodialysis or peritoneal dialysis and the particular embodiments thereof, medicaments administered during the treatment, and the dose of said medicaments, apparatuses used for treatment, such as disposable medical items such as tubes, bags, filters, such as dialysis filters or adsorption filters, and all treatment-related actions performed during a specific treatment, such as, for example, active change of position of the patient, manual interventions performed, such as massages, exercises performed by the patient, for example with an ergometer or other circulation-assisting exercises.

A medical treatment regime covers any action performed on, or by, a patient, any medicament administered to a patient, and any apparatus related to a specific treatment on a specific patient.

The present disclosure is particularly suitable for documenting the medical treatment of dialysis. In general, however, the disclosure can be used for documenting all types of medical treatments.

### BRIEF DESCRIPTION OF DRAWINGS

FIG. 1 shows a flowchart of an example method.

FIG. 2 shows a first exemplary view of a user interface of a software-controlled device.

FIG. 3 shows a second exemplary view of a user interface of a software-controlled device.

FIG. 4 shows a third exemplary view of a user interface of a software-controlled device.

FIG. 5 shows a fourth and fifth exemplary view of a user interface of a software-controlled device.

### DETAILED DESCRIPTION

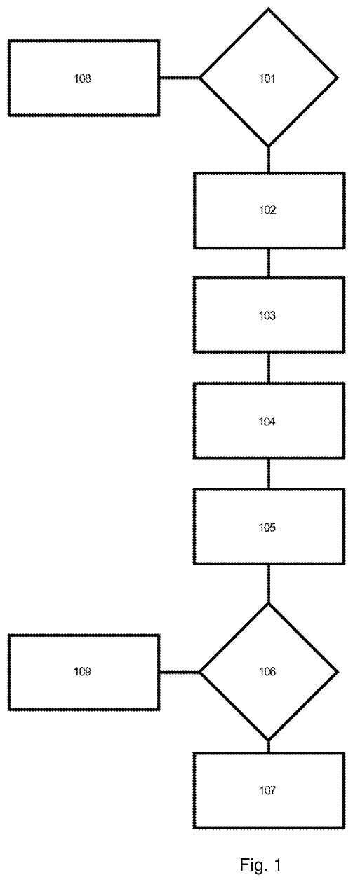

FIG. 1 shows a flowchart of an example method.

In step 101, a first authentication feature that has been input into a software-controlled device is checked, as described in WO 2021/048192 A1, for example, in which the software-controlled device is embodied as a tablet computer in one embodiment. The authentication feature can be any feature that characterizes and uniquely authenticates a first person. Such an authentication feature can be an input identification number known only to the first person (PIN), or a biometric feature of the first person that is captured with an appropriate sensor, such as for example a fingerprint, the facial image of the first person, an iris image of the first person, a captured gesture linked to the first person or a voice sample of the first person. Any feature that uniquely authenticates a specific person is suitable for this purpose.

The check on the first authentication feature includes whether the first authentication feature corresponds to an authentication feature from a first group of authentication features authorizing the input of information relating to the monitored medical treatment regimes into the user interface of the software-controlled device, and whether the first authentication feature is associated with a particular first person.

In general, the various configurations of the authentication features for groups of people can be stored in the software-controlled device itself and/or in a remote entity, for example a server computer, to which the software-controlled device is connected for data purposes via a network. Similarly, the check on the input authentication feature can take place locally, that is to say on the software-controlled device itself, and/or on a remote entity, the result of the check being transmitted to the software-controlled device by data transmission.

There are two groups of authentication features characterizing persons with different rights. The two groups can each include one or more persons, and individual authentication features can belong to one or both groups. Thus, two groups of authentication features should thus not necessarily be understood to mean two separate databases within the context of the present disclosure. The two groups are also formed, within the context of the present disclosure, by stored authentication features of two different people.

The present disclosure provides for authorizing a single person to input information relating to the monitored medical treatment regimes into the user interface of the software-controlled device and to instigate permanent storage of a confirming endorsement, but not both for a single software-controlled device.

In some examples, the first person and the second person are different people. The authorization to instigate the permanent storage of the confirming endorsement can follow automatically from the fact that the second input authentication feature characterizes a different person from the first input authentication feature. Accordingly, the authorization to instigate the permanent storage of the confirming endorsement does not necessarily have to be linked to an authentication feature and stored. According to the present disclosure, this authorization can also be derived from the fact that the second input authentication feature characterizes a different person from the person characterized by the first input authentication feature. This can be advantageous if a group of authentication features is stored that are associated with persons who are authorized both to input information relating to the monitored medical treatment regimes into the user interface of the software-controlled device and to instigate the permanent storage of the confirming endorsement that has been input.

In one embodiment, step 101 takes place after the software-controlled device is switched on, which means that general access to the software of the software-controlled device is also granted or prevented. In an alternative or additional embodiment, whenever the software is locked for use, step 101 takes place to unlock the software and make it accessible for a first authorized person

If it is established in step 101 that the input authentication feature corresponds to an authentication feature from the first group of authentication features, the user interface of the software-controlled device is enabled for the input of information relating to the medical treatment regime in step 102. Otherwise, the user interface of the software-controlled device is blocked for the input of information relating to the medical treatment regime, and an appropriate message can be prompted in step 108 to re-input the first authentication feature, for example by outputting an appropriate message by means of the user interface of the software-controlled device.

In step 103, information relating to the medical treatment regime is input into the software-controlled device.

By way of example, the mobile software-controlled device can be designed to automatically capture data relating to relating to the medical treatment regime. Alternatively, or additionally, such data can also be input into the user interface of the software-controlled device by means of manual input by a user. In this regard, reference is made to WO 2021/048192 A1, which shows a system including treatment apparatuses designed for dialysis and a mobile software-controlled device, depicted as a tablet computer in one embodiment, into which such data are manually input or which captures at least some of such data in automated fashion.

To capture and input the information relating to the medical treatment regime within the context of the present disclosure, there can be provision, as in WO 2021/048192 A1, for example for the software-controlled device to use a camera to capture a graphical code (such as a quick response (QR) code), which codes such data that can be displayed on the display of a dialysis device. A piece of software appropriately executed can be used to decode the graphical code to obtain the data relating to the associated medical treatment regime.

As such, for example a QR code printed on a medicament package can be captured and decoded in order to obtain information relating to the employed medical treatment regime and to transfer said information to the user interface of the device without manual input.

Such automatic detection of information relating to the medical treatment regime is not limited to administered medicaments or graphical codings, but can also include image evaluation software that detects a medical treatment regime within the context of the present disclosure on the basis of a camera signal.

In an alternative or additional embodiment, sensors for capturing radio signals such as radio frequency identification (RFID) or near-field communication (NFC) signals can also be used for this purpose. By way of example, disposable medical items can be recognized from their mere appearance. However, characteristic RFID tags can also be put onto the disposable items or the administered medicaments, which are detected by the software-controlled device if it includes appropriate sensors therefor. It is likewise possible for a piece of image recognition software to include text recognition and thus to capture a medical treatment regime by recognizing text printed on medicaments, for example.

If the medical treatment of a patient is continuously monitored using a camera of the software-controlled device, image recording and image or video evaluation software can also automatically capture medical actions on the patient that are not linked to apparatuses. Such medical actions are, for example, changes of position of the patient, massages, circulation-assisting activities and the like, as described herein.

The information relating to medical the medical treatment regime can also be input into the software-controlled device manually by means of text input into a user interface or by means of recorded voice. In one embodiment, voice recognition software can convert the spoken information into a text input.

In step 104, at least one confirming endorsement linked to the input information is entered.

The information relating to the medical treatment regime that is input into the user interface of the software-controlled device manually or automatically is linked in step 104 to a confirming endorsement that is input into the user interface manually. Such a confirming endorsement can be, for example, an activated button on a graphical user interface. Likewise, there can be provision for any information relating to the monitored medical treatment regime to have an associated activable button on the graphical user interface, for example a blank box that is filled with a color or provided with a touch activated tick. In some embodiments, the confirming endorsement is input by tapping on an appropriately labelled button on a touchscreen display of the software-controlled device. The result is that a graphical mark (such as a confirmation tick) can then signal that the input has been made.

To increase security, there can be provision for the input of the confirming endorsement to prompt further confirmation with an authentication feature of the first person (not shown in FIG. 1).

The manual input of the confirming endorsement confirms that the previously input information relating to the medical treatment regime is correct. A human inspection of the input data therefore takes place.

Following input of the confirming endorsement in step 104, a prompt to input a second authentication feature can be provided in step 105.

In step 106, the check on a second authentication feature that has been input into the software-controlled device on the basis of this prompt or on a second authentication feature that has been input into the software-controlled device unprompted takes place. Analogously to step 101, the check on the second authentication feature includes whether the second authentication feature corresponds to an authentication feature from a second group of authentication features authorizing the instigation of the permanent storage of the confirming endorsement, and whether the second authentication feature is associated with a second person.

The embodiments of the authentication feature for the second person can be the same as for the first person.

If it is established in step 106 that the input authentication feature corresponds to an authentication feature from the second group of authentication features, the confirming endorsement linked to the input information is permanently stored in step 107. Otherwise, an appropriate message prompting re-input of the first authentication feature can be displayed in step 109.

As a result of the permanent storage of the confirming endorsement linked to the medical treatment regime, even after deactivation of the software-controlled device it is possible to determine that a linked confirming endorsement has been input for a specific medical treatment regime that was input. This permanent storage can take place in the software-controlled device itself, for example, and/or by means of transmission to a remote entity, for example a server computer, or by data transmission via a network.

If the second authentication feature is not input, or the input second authentication feature does not meet the second condition, the input confirmation feature is not permanently stored, which means that after the deactivation of the software-controlled device it is no longer possible to determine that a confirming endorsement has been input. In one embodiment, if the second authentication is missing or incorrect, it is also possible to store an indication that the input information relating to the monitored medical treatment regime was provided with a confirming endorsement but that said confirming endorsement was not stored by means of the input of an authentication feature of an authorized second person. This can be done by means of a text message or by means of a graphical representation, such as for example a box that is only half filled or a tick that is only half completed.

In some embodiments, the confirming endorsement that has been input can be automatically deleted if no or an incorrect second authentication feature is input within a predetermined period after the input of the confirming endorsement. In such cases, the confirming endorsement can be to be input again after re-input of a valid first authentication feature.

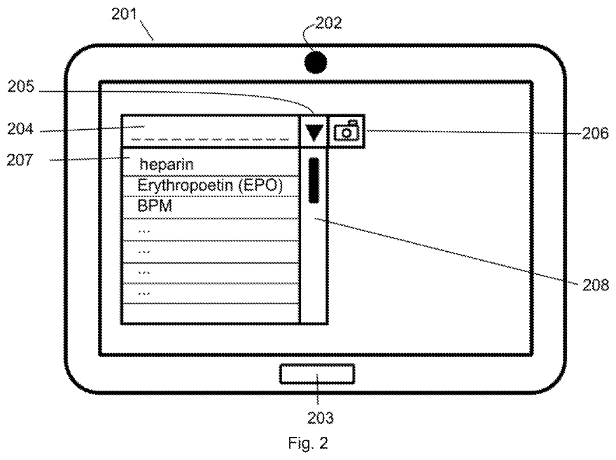

As an example of an embodiment of a software-controlled device, FIG. 2 shows a tablet computer on which an exemplary view of a user interface is shown. In this example, the user interface is displayed on a touchscreen display as an embodiment of a user interface. The user interacts with the user interface by touching appropriate buttons on the touchscreen display. Any other software-controlled devices, such as for example a smartphone, smartwatch or laptop or notebook computer, are likewise possible. Information can also be input into the user interface by using apparatuses other than a touchscreen display. The software-controlled device 201 can have at least one camera 202. Such devices are often equipped with two cameras on opposite sides of the device. Additionally, the software-controlled device 201 may include a fingerprint sensor 203.

In FIG. 2, the touchscreen depicts how medical treatment regimes can be input into the user interface of the software-controlled device 201. To this end, a medical treatment regime can be input into the field 204. Multiple options for inputting an applicable medical treatment regime are contemplated within the scope of the present disclosure.

By way of example, plain text can be input into the field 204, for example, by tapping on this field. Accordingly, a keypad (not depicted) can be displayed. Alternatively, tapping on the field 205 allows a selection menu 207 to be displayed, in which various medical treatment regimes are displayed so they can be selected (by tapping). FIG. 2 displays three medical treatment regimes in exemplary fashion, namely heparin and erythropoietin (EPO) as examples of medicaments and BPM (blood pressure measurement) as an example of a medical action on the patient. The field 208 contains a slide bar in order to move the displayed list in the field 208 upward or downward to display other entries.

Another method for inputting a medical treatment regime involves detecting said medical treatment regime automatically by means of image recognition. To this end, the field 206 with the camera symbol can be selected. Such a selection results in the software-controlled device activating an integrated camera function. The user then points the activated camera at a QR code, for example, that is attached to an administered medicament, as a result of which an applicable QR code detection and decoding function automatically transfers the information coded in the QR code, for example name and volume of the medicament, to the field 204. The automatic input of the medical treatment regime is not limited to a camera function and graphical codings. Alternatively, image and text recognition, and also sensors for capturing radio signals such as RFID or NFC signals, can be used. Accordingly, a blood pressure cuff on a patient can also be detected by image recognition software, for example, and accordingly “BPM” can be transferred to the field 204.

The user can then select the medical treatment regime displayed in the field 204, for example, by tapping and can thus input it into the software-controlled device 201.

The medical treatment regime can be linked to other data, for example to a medical treatment device (for example serial number of a dialysis machine), place of the medical treatment regime, name or synonym of the patient (patient number), date and time of the administration, etc.

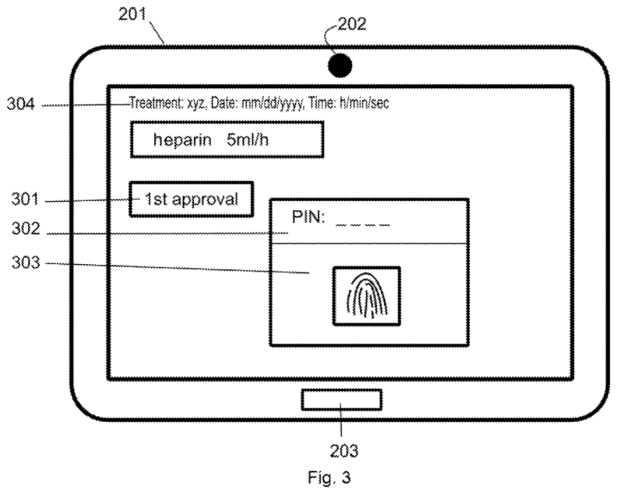

FIG. 3 depicts an example of a display 304.

If a medical treatment regime was input into the software-controlled device 201, this medical treatment regime can be provided with a first confirming endorsement. This is initiated in FIG. 3 by tapping on the field 301. To provide the medical treatment regime with a confirming endorsement, the input of a first authentication feature can be prompted. This is depicted in FIG. 3 by the fields 302 and/or 303. A PIN of a first person can be input in the field 302 in one embodiment. Alternatively, or additionally, the first person can authenticate himself by inputting a biometric feature. This is shown in FIG. 3 by virtue of the field 303 being selected and the fingerprint of the first person subsequently being detected using the fingerprint sensor 203. Alternative or additional sensors for biometric features are likewise possible.

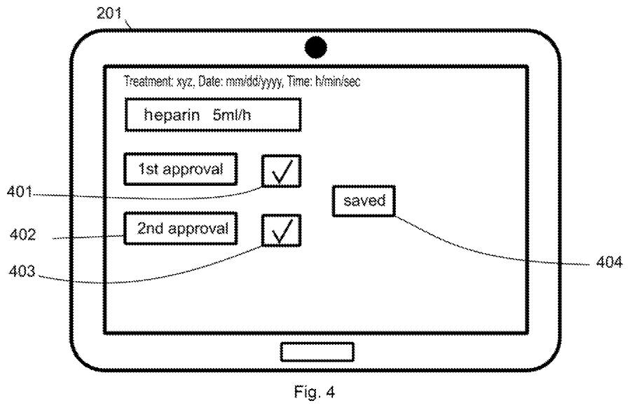

If the input first authentication feature meets the aforementioned first conditions, the successful first authentication can be depicted graphically. This is shown in FIG. 4 by the tick in the field 401. If the input first authentication feature does not meet the aforementioned first conditions, an error message (not shown) can be displayed and re-authentication can be prompted.

To permanently store the confirming endorsement, at least one other person can authenticate himself on the software-controlled device. Accordingly, in the embodiment shown in FIG. 3, the above operations can be repeated for another person. The security of these operations is increased if other people authenticate themselves on the software-controlled device 201 in the same way.

If the input second authentication feature meets the aforementioned second conditions, the successful second authentication can be depicted graphically. This is shown in FIG. 4 by the tick in the field 403. If the input second authentication feature does not meet the aforementioned second conditions, an error message (not shown) can be displayed and re-authentication can be prompted.

Following successful first and second authentication, the confirming endorsement is permanently linked to the medical treatment regime and permanently stored. This is shown in FIG. 4 by the field 404.

This linking can be reported to another device, for example a server computer, by the software-controlled device by means of data transmission via a network. This allows data backup to take place in the other device, and also allows for provisioning of the link information. In one embodiment, the remote device is unable to alter the linking. In some embodiments, specific people can change this linking to authenticate themselves. In some embodiments, these specific people are the first and/or the second person.

The input medical treatment regime can be stored in a blockchain together with the confirming endorsement confirmed by the dual authentication of two different people. The blockchain can store all formerly input data and information about the first and second persons authenticating themselves. Any change and addition to this information can therefore become trackable in a tamper-proof manner.

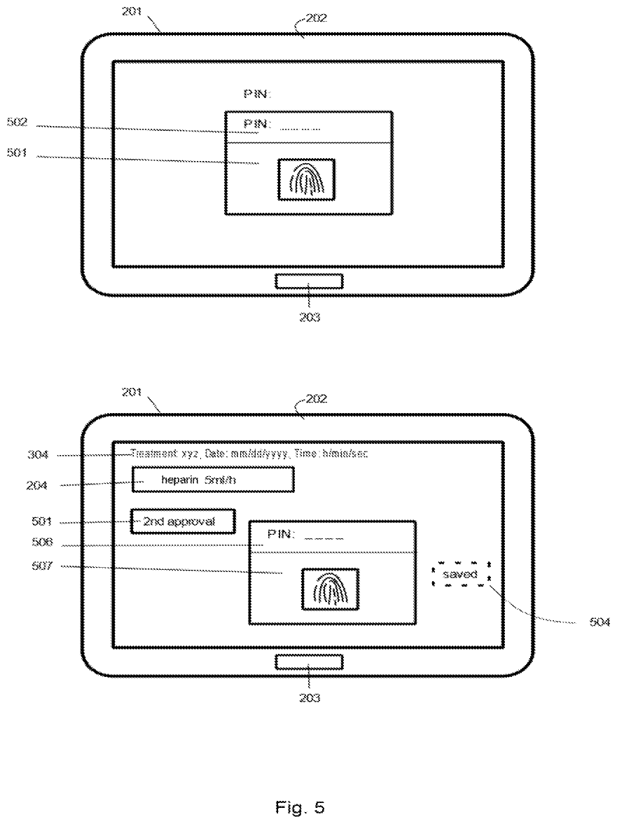

FIG. 5 depicts another embodiment of the present disclosure.

In contrast to FIGS. 2, 3 and 4, at the top of FIG. 5 the first authentication feature is input directly after the software-controlled device is switched on, or after the software-controlled device has been locked for use. The user inputs a first authentication feature in the field 501 and/or 502, as described herein. A check is then performed to determine whether the first authentication feature corresponds to an authentication feature from a first group of authentication features authorizing the input of information relating to the medical treatment regime into the user interface of the software-controlled device, and whether the first authentication feature is associated with a first person.

If the first check is successful, the software-controlled device is enabled for use and therefore also for the input of information relating to medical treatment regimes.

In the example depicted at the bottom of FIG. 5, the first user has input a medical treatment regime into the field 204. The input can take place in any manner described herein. The input of a confirming endorsement is initiated by tapping on the field 501 in this exemplary embodiment, after which the input of a second authentication feature in the field 506 and/or 507 (PIN or fingerprint) is prompted.

If the input second authentication feature meets the aforementioned second conditions, the successful second authentication can be depicted graphically. This is shown in exemplary fashion in FIG. 5 by the dashed field 504, which can be depicted in solid form after successful authentication, for example. As with the embodiments shown in FIGS. 2, 3 and 4, the confirming endorsement is subsequently linked to the medical treatment regime and permanently stored.

Aspects of the operations shown in FIG. 5 can readily be combined with other operations and features described with reference to FIGS. 2, 3 and 4.

The embodiment shown in FIG. 5 differs from the embodiments described in FIGS. 2, 3 and 4 in the time of the input first authentication feature, namely after the software-controlled device 201 is switched on or after the software-controlled device 201 is locked. In the example depicted in FIG. 5, a confirming endorsement that has been input is linked to a medical treatment regime and permanently stored after the input and checking of two authentication features that meet the aforementioned conditions and come from two different people (thus adhering to the 4-eyes security principle).

The present disclosure thus provides a higher level of security for the documentation of medical treatment regimes. The security can be increased by expanding the described techniques to three or more different people.

1-15. (canceled)

16. A method for securely documenting medical treatment regimes, the method comprising:
enabling a user interface of a software-controlled device for input of information relating to medical treatment regimes only if at least one first authentication feature is input into the software-controlled device and the first authentication feature meeting first conditions;
receiving information relating to a medical treatment regime into the software-controlled device;
receiving at least one confirming endorsement and linking the confirming endorsement to the input information; and
permanently storing the linked confirming endorsement only if at least one second authentication feature is input into the software-controlled device, wherein:
the second authentication feature meets second conditions,
the first conditions comprise the first authentication feature corresponding to an authentication feature from a first group of authentication features authorizing the input of information relating to the medical treatment regimes into the software-controlled device,
the first conditions further comprise the first authentication feature being associated with a first person,
the second conditions comprise the second authentication feature corresponding to an authentication feature from a second group of authentication features authorizing instigation of the permanent storage of the confirming endorsement, and
the second conditions further comprise the second authentication feature being associated with a second person.

17. The method of claim 16, wherein:
a sensor detects application of a particular medical treatment regime,
the detected medical treatment regime is communicated to the software-controlled device and is displayed on the user interface of the software-controlled device, and
the input of information relating to the medical treatment regime comprises receiving a selection the displayed medical treatment regime.

18. The method of claim 17, wherein:
the sensor is a camera, and
the application of the medical treatment regime to a patient is detected by a camera signal being communicated from the camera to image or video evaluation software that is configured to detect an application of a specific medical treatment regime to a patient on the basis of the camera signal.

19. The method of claim 17, wherein:
the sensor comprises a sensor for capturing graphical codings,
at least one medicament or apparatus linked to a medical treatment has characteristic graphical codings, and
the application of a medical treatment regime to a patient is detected by the at least one medicament or apparatus being held up to the sensor such that the sensor detects the characteristic graphical codings.

20. The method of claim 17, wherein:
the sensor comprises a sensor for capturing radio signals that include radio frequency identification (RFID) signals or near-field communication (NFC) signals,
at least one medicament or apparatus linked to a medical treatment is provided with an apparatus for transmitting characteristic radio signals, and
the application of a medical treatment regime to a patient is detected the at least one medicament or apparatus being held up to the sensor so the sensor detects the characteristic radio signals.

21. The method of claim 16, wherein the first authentication feature and second authentication feature comprise at least one of a personal identification number (PIN), a fingerprint, a personal signature, an iris image, a facial image, a gesture, or a voice sample.

22. The method of claim 16, further comprising storing the input information, wherein the stored information is used to derive therapy recommendations based on the stored endorsement.

23. The method of claim 16, further comprising transmitting the stored endorsement to a further entity, which is remote from the software-controlled device, by remote data transmission.

24. A software-controlled device configured to perform operations comprising:
enabling a user interface of the software-controlled device for input of information relating to medical treatment regimes only if at least one first authentication feature is input into the software-controlled device and the first authentication feature meeting first conditions;
receiving information relating to a medical treatment regime into the software-controlled device;
receiving at least one confirming endorsement and linking the confirming endorsement to the input information; and
permanently storing the linked confirming endorsement only if at least one second authentication feature is input into the software-controlled device, wherein:
the second authentication feature meets second conditions,
the first conditions are based on the first authentication feature corresponding to an authentication feature from a first group of authentication features authorizing the input of information relating to the medical treatment regimes into the software-controlled device,
the first conditions are further based on the first authentication feature being associated with a first person,
the second conditions are based on the second authentication feature corresponding to an authentication feature from a second group of authentication features authorizing instigation of the permanent storage of the confirming endorsement, and
the second conditions are further based on the second authentication feature being associated with a second person.

25. The software-controlled device of claim 24, wherein the software-controlled device comprises a portable computer, tablet computer, smartphone or smartwatch.

26. The software-controlled device of claim 24, wherein:
a sensor detects application of a medical treatment regime,
a detected medical treatment regime is communicated to the software-controlled device and is displayed on the user interface of the software-controlled device, and
the input of information relating to the medical treatment regime comprises selecting the displayed medical treatment regime.

27. The software-controlled device of claim 26, wherein:
the sensor is a camera, and
the application of a medical treatment regime to a patient is detected by virtue of the camera signal being communicated to image or video evaluation software that is configured to detect an application of a specific medical treatment regime to a patient on the basis of the camera signal.

28. The software-controlled device of claim 26, wherein:
the sensor comprises a sensor for capturing graphical codings,
at least one medicament or apparatus linked to a medical treatment has characteristic graphical codings, and
the application of a medical treatment regime to a patient is detected by virtue of the at least one medicament or apparatus being held up to the sensor so the sensor detects the characteristic graphical codings.

29. The software-controlled device of claim 26, wherein:
the sensor comprises a sensor for capturing radio signals that include radio frequency identification (RFID) signals or near-field communication (NFC) signals,
at least one medicament or apparatus linked to a medical treatment is provided with an apparatus for transmitting characteristic radio signals, and
the application of a medical treatment regime to a patient is detected by virtue of the at least one medicament or apparatus being held up to the sensor so the sensor detects the characteristic radio signals.

30. The software-controlled device of claim 24, wherein the first and second authentication features comprise at least one of a personal identification number (PIN), a fingerprint, a personal signature, an iris image, a facial image, a gesture, or a voice sample.

31. A system comprising at least one medical device designed to treat a patient and a computer-readable medium storing instructions that, when executed by at least one processor, cause the at least one processor to perform operations comprising:
enabling a user interface of the software-controlled device for input of information relating to medical treatment regimes only if at least one first authentication feature is input into the software-controlled device and the first authentication feature meeting first conditions;
receiving information relating to a medical treatment regime into the software-controlled device;
receiving at least one confirming endorsement and linking the confirming endorsement to the input information; and
permanently storing the linked confirming endorsement only if at least one second authentication feature is input into the software-controlled device, wherein:
the second authentication feature meets second conditions,
the first conditions are based on the first authentication feature corresponding to an authentication feature from a first group of authentication features authorizing the input of information relating to the medical treatment regimes into the software-controlled device,
the first conditions are further based on the first authentication feature being associated with a first person,
the second conditions are based on the second authentication feature corresponding to an authentication feature from a second group of authentication features authorizing instigation of the permanent storage of the confirming endorsement, and
the second conditions are further based on the second authentication feature being associated with a second person.

32. The system of claim 31, further comprising an entity, which is remote from the software-controlled device and which exchanges data with the software-controlled device.

33. The system of claim 32, wherein the medical device is a blood treatment device.

34. The system of claim 31, wherein the stored endorsement is used to derive therapy recommendations.

35. The system of claim 31, wherein the first and second authentication features comprise at least one of a personal identification number (PIN), a fingerprint, a personal signature, an iris image, a facial image, a gesture, or a voice sample.
